(19) United States
(12) Patent Application Publication
(10) Pub. No.: US 2020/0284713 A1
(43) Pub. Date: Sep. 10, 2020

(54) METHOD FOR DEFORMING DEFORMABLE BODIES, AND DEVICES FOR THIS PURPOSE

(71) Applicant: Universtat Greifswald, Greifswald (DE)

(72) Inventors: OLIVER OTTO, Greifswald (DE); Fabian Czerwinski, Stralsund (DE); Bob Fregin, Greifswald (DE)

(21) Appl. No.: 16/649,608

(22) PCT Filed: Sep. 21, 2018
(86) PCT No.: PCT/EP2018/075605

§ 371 Date: Mar. 20, 2020

(30) Foreign Application Priority Data
Sep. 23, 2017 (DE) 10 2017 008 946.9

(51) Int. Cl. G01N 15/14; B01L 3/00; G01N 33/50

(52) U.S. Cl. CPC G01N 15/1404; B01L 3/502776; G01N 2015/1411; G01N 15/1463; B01L 2200/0636; G01N 33/5094

(57) ABSTRACT

A method for deforming deformable bodies, preferably droplets or cells, comprising feeding a sample fluid into a microfluidic channel to create a laminar flow of the sample fluid, wherein the sample fluid transports deformable bodies, and feeding a sheath fluid into the microfluidic channel to create a laminar flow of the sheath fluid such that the sheath fluid directly borders the sample fluid in a border region of the microfluidic channel and flows in the same direction as the sample fluid at least in the border region. The viscosity of the sheath fluid is greater than the viscosity of the sample fluid, and the average flow rate of the sample fluid is greater than that of the sheath fluid.

### TECHNICAL FIELD

The present invention relates to a method for deforming deformable bodies, in particular droplets or cells, and a device for this purpose. It further relates to a method for determining the mechanical properties of deformable bodies, preferably cells.

### PRIOR ART

The analysis of biological cells is often achieved by the selective binding to the cell of various fluorescent dyes. One possibility is to bind dyes to antibodies. These dyes bind to surface proteins if these are present on a cell. The cell is then marked with this dye. The respective cell types which are searched for can thus be detected by means of a suitable microscope, even in heterogenous samples.

In the case of cell suspensions, it is customary to use the flow cytometer developed by Dittrich and Gohde in 1968 which is described in DE 1 815 352. In this process, the cells are examined individually one after the other for the presence of dyes. At the same time, it is also possible to sort the cells according to measurement results (Orfao A. et al., Clin. Biochem. (1969), Tung Y. C. et al., Sens. Actuators, B (2004)). Magnetic nanoparticles with corresponding antibodies are also used as a marker so that cells can be sorted by magnetic fields (referred to as MACS).

Over the last few decades, technologies have also become established shifting the focus of the analysis from molecular to mechanical characterisation of the cell. These approaches are based on classifying sequences occurring due to changes in the dynamic polymer network of the cell (cytoskeleton). The cytoskeleton spans the entire cell and is of particular importance for a number of key cell processes. Unlike molecular methods, a mechanical characterisation of the cell allows an analysis not involving hypotheses, in other words an analysis based on an inherent marker.

Various technologies are currently available for mechanical analysis, namely atomic force microscopy, laser-based force spectroscopy and micropipettes. These methods determine the mechanical properties of cells based on deformation under a predefined force. It is possible to analyse roughly 100 cells per hour, which means that respective methods are suitable for smaller cell populations. However, it is not possible to transfer this method to the applied research owing to the low throughput rates.

A further development of these technologies is hydrodynamic deformation. In 2015, a method was published in the form of real-time deformability cytometry (RT-DC) which allows a mechanical characterisation of cells in real time at a rate of up to 1000 cells per second (O. Otto et al., Real-Time Deformability Cytometry: On the Fly Mechanical Phenotyping. Nature Methods, 12, 199-202 (2015) and WO 2015/024690 A1).

In RT-DC, cells are pumped through a microfluidic channel using a syringe pump. This channel has a diameter that is a few percent larger than the respective cell diameter. Owing to hydrodynamic interaction, the shear forces and normal forces deform the cells in the channel. RT-DC has proven advantageous in responding to basic questions arising in life sciences and allows a number of applications which are impossible either with classic flow cytometry or with classic mechanical methods. Reference is made to WO 2015/024690 A1 as regards details of this technology.

### Technical Object

The inventors became aware of various problems in the prior art.

Weaknesses with the methods already mentioned, methods which are antibody-based, are, for example, that the cell-specific surface protein must be known for the marker. Furthermore, it is necessary to pre-treat the cell suspension with comparably expensive fluorescent dyes. In addition, the properties of the cells change due to foreign substances (such as the dyes) and the cell suspensions are also contaminated with foreign substances.

The latter can lead to the fact that these suspensions can no longer be used in patients afterwards and must be disposed of, as the dyes may in some cases be harmful to patients.

In addition, this method does not allow non-destructive analysis of cells, as the cells are marked with the antibodies and are therefore no longer “natural”, which means that they will no longer be available for further examinations or applications.

The inventors became furthermore aware that the existing RT-DC systems present the disadvantage that the microfluidic channels always have to be adapted to the cell size. The channel must always be slightly larger than the diameter of the largest cell to be examined: If the diameter of the channel is too small, the cell will get “stuck”, whereas if the diameter of the channel is significantly larger than the diameter of the cell, no notable shear forces will act on the cell due to the hydrodynamics; in other words, the cell will not be deformed, or will only be deformed to a minor extent. If a cell “gets stuck”, this is a significant disadvantage, as the rest of the sample can in many cases no longer be used. Often the entire volume of the sample is only a few μl, and as this sample volume fills the entire chip and the chip has to be replaced in the event of a cell getting stuck, the entire sample volume (or a major part thereof) is consequently thrown away. As the samples are often very difficult to obtain, this is a major problem and a non-negligible cost factor.

In the prior art in relation to RT-DC, this leads to the fact that the microfluidic chips may have to be replaced during a series of measurements, resulting in contamination and dips in the throughput of samples. Furthermore, measurements carried out in chips of various sizes during RT-DC are not always comparable. This is due to the fact that the forces are scaled to the cells due to the size of the cells and the cross-section of the channel. To this extent, there are two scaling variables, which can be adjusted independently of each other. If one of these variables (e.g. the size of the channel) changes, the results are consequently not comparable. Furthermore, some cells cannot currently be measured, as the channels cannot be made small enough.

In addition, the channel size is limited by the manufacturing process of the microfluidic chips. Specifically, for example, the plastic material from which the channel is made can only guarantee stable channel geometries up to a minimum height of a few micrometres. This is due to the fact that these chips are currently manufactured by means of soft lithography. These methods are very cost-intensive and are described in O. Otto et al., Real-Time Deformability Cytometry: On the Fly Mechanical Phenotyping. Nature Methods, 12, 199-202 (2015). The high costs prohibit the method from being used efficiently in application-based research. Another manufacturing method would involve injection moulding, although a negative mould must be provided. As a result, the minimum size of the moulds which are to be designed is limited. Furthermore, the initial costs of such a method tend to be high, as the negative mould is very expensive.

To achieve a combination of fluorescence measurement, sorting and mechanical analysis, using the RT-DC microfluidic chips on its own is not sufficient, as the samples can manifest the difficulties (blockage and adaptation of the chip size to the sample size) described above. It is therefore difficult to incorporate RT-DC into the flow cytometer according to DE 1 815 352 developed by Dittrich and Gohde.

### DESCRIPTION OF THE INVENTION

The aim of the invention is to mitigate the problems mentioned above and in particular the problems with existing RT-DC systems. In particular, an object of the invention is to adapt the measurements to channel geometries, measuring systems and sample specifications without having to make any major direct, physical reconfigurations or alterations to the measuring equipment. In addition, the intention, in some embodiments at least, is also to allow characterisation and, where applicable, sorting in a flow cytometer. The cuvettes used in flow cytometers typically have a diameter of several 100 μm, which is too large for mechanical cell measurement, as the forces exerted on the cells would be much too small. An aim of the present invention is to also be able to trigger deformations of cells with cuvettes with such diameters and to be able to perform measurements of mechanical properties of these cells.

The invention is defined by claim 1. Preferred embodiments are defined by the dependent and subsidiary claims.

A method according to the invention is a method for deforming deformable bodies, which can be droplets or cells, for example. A deformable body is a body which can be transported in a flowing fluid (preferably: liquid) and can be deformed by the shear forces occurring in such a flow or by the forces occurring at the boundary layer between the fluids.

The method according to the invention comprises a step of feeding a sample fluid into a channel so that a laminar flow is created. This sample fluid is a fluid which contains and transports deformable bodies. These deformable bodies are contained in the sample fluid in the form of a suspension or slurry and are therefore transported together with the sample fluid as it flows through the channel.

The channel is preferably a microfluidic channel. This is a channel with a diameter of significantly less than 1 mm and in particular with a channel diameter within a range of 100 nm to 500 μm. A laminar flow is understood to mean a flow in which there is no turbulence. Such a flow can be defined by the fact that the Reynolds number is significantly less than 1000.

According to the invention, a sheath fluid is further fed into the channel, in which the sample fluid flows. This sheath fluid is fed into the channel such that a laminar flow of the sheath fluid is created and such that the sheath fluid directly borders the sample fluid in a border region of the microfluidic channel. “Directly borders” is understood to mean that there is no physical separation between these two flows, in other words that the two flows border each other without any barrier and, in principle, can also merge into each other. The border region can encompass the entire length of the channel, but it is sufficient if the two fluids only directly border each other in a partial area of this channel, while they are separated from each other in other areas.

The sheath fluid can be any fluid (preferably: liquid) other than the sample fluid, although the two fluids will be defined in more specific detail later. The sheath fluid is preferably provided on two sides of the sample fluid, whereby this flow of the sheath fluid is preferably symmetrical with regard to the sample fluid. However, it is sufficient if the sheath fluid is on one side of the sample fluid. A symmetrical arrangement, in particular an arrangement whereby the sheath fluid surrounds the flow of the sample fluid, results in a symmetrical geometry which consequently also leads to a symmetrical deformation of the cells and therefore to a simpler and better analysis.

As regards the physics, cells or deformable bodies can generally be modelled as elastic objects which can be deformed due to shear and normal forces in narrow microchannels. For experiments in RT-DC, a comprehensive description of the forces acting on the bodies is given in A. Mietke et al., Extracting Cell Stiffness from Real-Time Deformability Cytometry: Theory and Experiment, Biophysical Journal 109, 2023-2036 (2015).

The key parameters determining the degree of deformation are the elasticity and viscosity (viscoelasticity) of the cells, the surface tensions of the cells used and the viscosities and surface tension of the sheath fluid and sample fluid, which are material to the scaling of the speed profile and therefore to the shear and normal forces. In RT-DC, the deformation of the cells (deformation D) is described as D=1−(2√(πA))/P, where A is the cross-sectional area (projection area) and P the circumference of the cells. However, this definition of deformation as “circularity” is just one example, and other definitions of deformation can also be used. Other definitions of deformation would be the geometrical moment of inertia and the axial ratio.

According to the invention, the dynamic viscosity of the sheath fluid is greater than the dynamic viscosity of the sample fluid. Dynamic viscosity here is understood to mean the viscosity as it occurs at the shear rates occurring in the two fluids in the border region of the channel. This can be achieved by the sheath fluid being either shear-thickening or a Newtonian liquid with a high viscosity, while the sample fluid is shear-thinning or alternatively a Newtonian liquid with a low viscosity. A shear-thinning liquid is preferred as the sample fluid, as it leads to a particularly advantageous behaviour of the liquids. However, it is important that the sample fluid always has a lower dynamic viscosity at the occurring shear rates than the sheath fluid at the occurring shear rates. This ensures that the sheath fluid forms a virtual channel for the sample fluid, as described in detail later. The shear rates typically occurring are within a range of 100 1/s to 10,000 1/s, and it is preferred that the sheath fluid and the sample fluid meet the stated conditions for the viscosities for all shear rates within this range. It is also possible that the sheath fluid and the sample fluid have the same viscosity at low shear rates and the sample fluid has shear dilution at higher shear rates.

In addition, it is preferred that the average flow rate of the sample fluid is higher than that of the sheath fluid. The average flow rate of the sample fluid is understood to mean that the flow rate is determined as an arithmetical mean of the flow rate in a cross-section perpendicular to the flow rate of the fluid in the microfluidic channel. The viscosity of the sheath fluid and the sample fluid respectively relates to the viscosity at room temperature as measured with an MC302 shear rheometer from Anton Paar, in which a cone-plate system with a 50 mm diameter, a 2 degree angle and a cylinder system is used.

Due to the feeding of two different fluids, the channel can be “virtually” reduced, unlike the technology described in WO 2015/024690 A1. That part of the channel in which the sample fluid flows forms the actual channel, in other words that part of the channel in which the deformable bodies are deformed in the parabolic flow profile owing to the laminar flow (Li et al., J. Fluids Eng. 2011, 111202). Contrary to this, the sheath fluid, so to speak, forms part of the boundary of the microfluidic channel as regards the flow properties of the sample fluid. In other words, this sheath fluid is comparatively “solid” in relation to the sample fluid, as it has a higher dynamic viscosity than the sample fluid at the present shear rate, which is why it acts in the same way as the wall of the microfluidic channel in WO 2015/024690 A1. This method of virtually changing the size of the channel is referred to as “virtual channel resizing”.

It is of advantage here that the virtual channel which is formed by the sample fluid 10 is microfluidic, in other words that it fulfils the definition of a microfluidic channel. However, the outer channel 12 can be significantly larger and does not have to be microfluidic. For example, it could be a cuvette.

The portion of the microfluidic channel in which the sheath fluid and sample fluid flow can accordingly be controlled by controlling the respective flow rates of the sheath fluid and the sample fluid. This leads to the fact that the channel width of the “virtual” microfluidic channel (which is defined by the sample fluid) can then be adjusted virtually without having to adjust the physical width of the actual channel separately. As a result, different cells can be examined with the same channel without having to use different channels for specific cell types.

To obtain the desired flow profile, the viscosities, flow rates and channel geometry can be adjusted with the help of Li et al., J. Fluids Eng. 2011, 111202, equation [20].

The dynamic viscosity of the sheath fluid is preferably within a range of 1 mPa s to 1 Pa s, even more preferably within a range of 50 mPa s to 250 mPa s, and/or the viscosity of the sample fluid is within a range of 1 mPa s to 100 mPa s, preferably a range of 5 mPa s to 50 mPa s. Corresponding ranges have proven particularly advantageous. The dynamic viscosities are within these shear rate ranges in the border region and preferably across the entire shear rate range of 100 1/s to 10,000 1/s.

The average flow rate of the sample fluid is preferably within a range of 0.1 cm/s to 1 m/s and/or the average flow rate of the sheath fluid is within a range of 0.1 cm/s to 1 m/s, but in any case below the average flow rate of the sample fluid. Corresponding preferred flow rates have proven to be particularly advantageous in tests. For the same reasons, it is also preferred that the flow rates (volumetric flow rate) of the sample fluid to the sheath fluid are within a range of 1:1 to 20:1. This was shown in experiments, although flow rates (volumetric flow rate) of the sample fluid to the sheath fluid of 1:1 to 1:20 are also possible, depending on the channel geometry.

It is also preferred that the sample fluid is a shear-thinning liquid containing the deformable bodies. Shear thinning is understood to mean that the viscosity of the sample fluid is reduced when a shear force is applied. This enables the sample fluid to flow more quickly, as it exerts a lower resistance. This is an advantage when it comes to implementing the method according to the invention.

It is also preferred that the sample fluid and sheath fluid consist of liquids which do not mix, or only do so to a minor extent, at least within the time scale during which the deformable bodies traverse the border region. In particular, this characteristic could be achieved by one of the liquids being polar, while the other liquid is apolar. Using respective liquids makes it easier to separate the liquids, thereby leading to simpler hydrodynamics and consequently to a better possibility of analysing the results.

Furthermore, it is preferred that the sheath fluid is a Newtonian liquid. A Newtonian liquid is understood to mean a liquid whose viscosity does not change at different shear rates, or only changes minimally. By using such a Newtonian liquid, it is possible to ensure that the mechanical properties of the sheath fluid only change slightly, if at all. Comparatively good and reliable results can be achieved with such a method as well. However, the sheath fluid can also be shear-thickening, thus further increasing the cited effect of forming a virtual channel.

It is also preferred that the method for determining the mechanical properties of deformable bodies, in particular cells, is used. The deformation of the deformable bodies is preferably measured by means of an optical method. In particular, the method as applied in WO 2015/024690 A1 can be used here. The mechanical properties of cells can thus be determined efficiently. WO 2015/024690 A1 also describes how the optical data can be evaluated to determine mechanical properties.

A further application of the method consists in the possibility of combining the method according to the invention with existing technologies for analysing cells with respect to their cell biology. It is thus possible using fluorescent dyes, for example, to mark organelles of cells or, for example, proteins in their cytoskeleton (e.g. actin). If the cells are then deformed using the method according to the invention, it can be observed how the cytoskeleton changes or how the organelles change. This allows a completely new analysis of cells.

In particular, such an analysis can be performed with the help of flow cytometry (known, for example, under the brand “FACS”). Such a method is described in A. Adan, Flow cytometry: basic principles and applications, Crit. Rev. Biotechnol., 37, 163-176, 2017, for example.

In principle it would be possible with RT-DC to install chips in a flow cytometer. However, it would be likely that these would cause a blockage. Furthermore, it would be impractical to have to use different chips depending on the cell size. Since the present technology is not confined to microfluidic channels, there is no such risk of blockage. It is therefore realistically possible to use such a method.

In addition, the method according to the invention can be used to sort deformable bodies, in particular cells. In principle, there are two sorting methods: active and passive sorting. With passive sorting, the properties of cells or generally deformable bodies are used to sort them. The inertia properties of deformable bodies, for example, can be used to sort them. Due to the use of a particular filter device, the bodies separate on their own into two populations, so to speak, i.e. without an external action. A simple example from everyday life regarding passive sorting would be a sieve. Again, no external action is required here to separate the large particles from the small particles: The latter fall through the sieve, while the former get caught in the sieve. One problem here, however, is that the properties of the bodies must be known in advance for this sorting, which is not always the case. In the example of the sieve, the typical sizes of the particles must be known, for example.

In the case of active sorting, the bodies to be sorted are actively moved to one or the other population. An example from everyday life would be, for example, the manual sorting of objects based on size. Unlike the aforementioned example of a sieve, external action is required here. However, in the case of deformable bodies and cells in particular, a complicated combination of microfluidics and control electronics is required for RT-DC here, which is extremely costly and cannot be scaled or is extremely difficult to scale.

As the present system can dispense with microfluidics, this complication is circumvented so that it can be used for cell sorting or sorting of deformable bodies. In other words, as the present system allows an integration of mechanical cell measurement into a flow cytometer, its sorting unit (e.g. based on piezo elements) can be used directly and without major modifications. Such a sorting unit exerts an impulse on the respective deformable body or cell so that this body/cell moves into a respective other reservoir. A flow cytometer (FACS) can in particular be used for active sorting of cells.

With the given channel geometries and materials for sample fluid and sheath fluid, the width of the flow of the sample fluid is adjusted by choosing a flow rate ratio. In addition, the width of the sample fluid also depends on the total flow rate. It is particularly preferred here that, prior to the start of deformation or measurement, the total flow rate of sample fluid and sheath fluid (in other words the total flow of liquid or fluid per time, i.e. the volumetric flow rate) is increased from an initial value to a final value or target value. The inventors noticed that the fluids often manifest hysteresis behaviour whereby the relative width of the flow of sample fluid with regard to the channel remains constant as the flow rate rises across a wide range of flow rates, whereas such behaviour does not occur to the same extent as the flow rate falls. As it is generally advantageous to keep a relative width of the flow of the sample fluid relative to the channel constant at different flow rates, and as a corresponding behaviour is only observed as the absolute flow rate or total flow rate rises, it is advantageous to increase the total flow rate, but not reduce it.

It is also preferred that a device designed to perform the method according to one of the preceding claims be provided. Such a device has in particular a control device and feeds for a sheath fluid and a sample fluid comprising the respective features.

### BRIEF DESCRIPTIONS OF THE DRAWINGS

FIG. 1 is a photo of a microfluidic channel for performing a method according to the invention. A laminar flow of sample fluid is channelled into a narrow channel through two flows, which are also laminar, of sheath fluid.

FIG. 2 is a top view of a section of the microfluidic channel. Bright field picture with transmitted light focusing on the narrow channel 12.

FIG. 3 shows the behaviour of the relative width of the flow of the sample fluid to the total width of the channel depending on the flow rate. The insets show images of the channel at different flow rates.

FIG. 4 shows the deformation behaviour of a leucocyte according to the prior art.

FIG. 5 shows the deformation behaviour of a leucocyte according to the method according to the invention.

FIG. 6(a) shows a scatter plot for leucocytes according to the prior art.

FIG. 6(b) shows a scatter plot of a leucocyte according to the method according to the invention.

FIG. 7 shows measurement results as regards the dynamic viscosities of the liquids used.

### DETAILED DESCRIPTIONS OF THE DRAWINGS

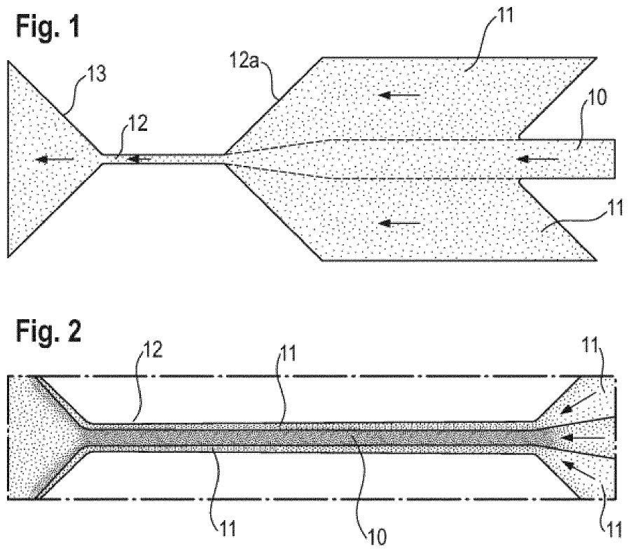

FIG. 1 shows a photo of the microfluidic chip used. The microfluidic channel 12 comprises an inlet 12a in which the sheath fluids 11 are fed from two sides of a sample fluid 10. The directions of flow within this channel are each depicted with arrows. As can be seen from FIG. 1, the two sheath fluids 11 surround the sample fluid 10 symmetrically. Once these sheath fluids 11 have traversed the microfluidic channel 12, they are discharged from the outlet 13.

A detailed view of the microfluidic channel 12 can be seen in FIG. 2. As is clearly evident here, the flow of sample fluid 10 is also clearly separated from the two side flows of sheath fluid 11 within the microfluidic channel 12. This is even visibly shown in a bright field picture, as can be seen in FIG. 2. To this extent, the flow of sample fluid 10 flows in a virtual channel within the microfluidic channel 12, which is delimited by the sheath fluid 11.

With regard to basic physical principles, it should be noted that in laminar flow systems, which are characterised by low Reynolds numbers (a Reynolds number of less than 1000), a flow forms along a channel in the direction of flow which has a parabolic speed profile perpendicular to the direction of flow. In particular, the edges of the channel are subject to the condition that the speed of the molecules must be 0. At the same time, the highest speed is achieved at points where the flow of molecules is least disturbed by the edges or other flows. For channels with a laminar flow, this is in the middle. At the boundary between the two fluids, the speed of these fluids is not zero.

As shown in FIG. 1, the sheath and sample fluids 11 and 10 flow from right to left, wherein the sheath fluids 11 channel the sample fluid 10. These fluids 10, 11 flow in a laminar formation through the microfluidic channel 12, which in the example shown in FIG. 2 measures 40 μm×40 μm in the cross-section perpendicular to the direction of flow. Within the narrow virtual microfluidic channel 12 through which the sample fluid 10 flows, the speed of the flow of sample fluid 10 increases to roughly 50 cm per second in the middle of the channel. The width of the virtual channel through which the sample fluid 10 flows depends on the flow rates transmitted by syringe pumps which provide the flow of the sheath fluid 11 and the sample fluid 10, depending on the given channel size and viscosities. Syringe pumps are advantageous, as they allow precise control of the flow volume with an accuracy of nl/s. In an RT-DC experiment, the ratio between the flow rates in the sheath fluid and sample fluid is adjusted so that the cells flow through the middle of the microfluidic channel 12. With virtual channel resizing, the sheath fluid in the present embodiment consists of a polymer solution (e.g. 100 mMol polyethylene glycol 8000 in a phosphatic buffer solution) which is significantly more viscous than the sample fluid. As shown, the flows only mix slightly, as can be clearly seen from the clear edges in FIG. 2. This was confirmed by finite element simulations. Owing to the flow time of the two fluids, there was no diffusion or only minimal diffusion.

In the present embodiment, virtual channel resizing is performed as follows: The composition of the sheath fluids 11 is selected so that they have a significantly elevated viscosity compared to the sample fluid 10, although the shear rate has little bearing on this. The first experiments were carried out with polyethylene glycol (PEG) 8000 fully dissolved with a concentration von 100 mMol in a PBS (phosphate-buffered saline solution). PBS not containing notable quantities of calcium and magnesium was used. However, another liquid can, in principle, also be used. The solution has a dynamic viscosity of roughly 235 mPa s (at a shear rate of 1 1/s-10,000 1/s). This viscosity was measured with an MC302 rheometer from Anton Paar with a cone-plate system with a diameter of 50 mm, an angle of 2 degrees and with a cylinder system. The solutions in the sample flow were measured with a rolling ball viscometer (Anton Paar, Lovis2000-DMA). The flow rates at the edge of the channel are then reduced by a factor of more than 20. An analytical description of this behaviour can be found in J. Li, P. S. Sheeran, C. Kleinstreuer, Analysis of Multi-Layer Immiscible Fluid Flow in a Microchannel, J. Fluids Eng. 133, 111202 (2011).

The parabolic profile of the flow of sample fluid 10 is formed by means of a virtually smaller channel which now exists owing to the boundary area between the sample fluid 10 and the sheath fluids 11. To this end, the changes in shear rate perpendicular to the direction of flow in the flow of the sample fluid become greater compared to the RT-DC according to WO 2015/024690 A1. This causes greater shear and normal forces in the sample fluid 10 than was the case in previous RT-DC tests. The flow rates or chips could also be changed here, but this would reduce the comparability of the results. In addition, disadvantages such as frequent chip changes and/or blockages of the channels are also avoided.

The following preferred conditions for the composition of the sheath fluids 11 result from the aforementioned observations: It is of advantage if the composition of the materials and solutions for the sample fluid 10 and the sheath fluids 11 are selected so that these fluids 10, 11 do not mix—owing to diffusion—in the time scale during which the deformable objects are being deformed and flow through the channel.

It is further of advantage if the material/solution of the sheath fluid 11 has a higher viscosity than the material/solution of the sample fluid 10. However, it is also conceivable that the sheath fluid and sample fluid have the same viscosity at low shear rates, but the sample fluid is shear-thinning. The viscosity of the sheath fluid 11 should depend as little as possible on the shear rate (in other words, it should be Newtonian) or be shear-thickening. For PEG 8000 (100 mMol) in PBS, for example, the dynamic viscosity (measured with a rheometer) is approximately 235 mPa s over a shear rate range of 1 per second to 10,000 per second.

It is further of advantage if the material/solution of the sample fluid is shear-thinning. For methyl cellulose (0.5%) in PBS, the dynamic viscosity follows a power law with an exponent of 1 to 0.677 (Herold, ArXiv 2017, https://arxiv.org/ftp/arxiv/papers/1704/1704.00572.pdf).

Successful realisations of virtual channel resizing can be achieved with different conditions. This was shown in experiments using the compositions of sample fluid and sheath fluid listed in the table below:

| Successful | | |
|---|---|---|
| realisation | Sample fluid material | Sheath fluid material |
| 1 | Methyl cellulose (0.5% w/v) | Polyethylene glycol 8000 |
| | in PBS, dynamic viscosity | (100 mM) in PBS, dynamic |
| | 14 mPa s, shear-thinning, | viscosity 235 mPa s, |
| | 285 mOsm | almost Newtonian, >2800 |
| | | mOsm |
| 2 | Methyl cellulose (0.7% w/v) | Polyethylene glycol 8000 |
| | in PBS, dynamic viscosity | (100 mM) in PBS, dynamic |
| | 21 mPa s, shear-thinning, | viscosity 235 mPa s, |
| | 289 mOsm | almost Newtonian, >2800 |
| | | mOsm |
| 3 | Methyl cellulose (0.5% w/v) | Polyethylene glycol 8000 |
| | in PBS, dynamic viscosity | (20 mM) in PBS, dynamic |
| | 14 mPa s, shear-thinning, | viscosity 18 mPa s, almost |
| | 285 mOsm | Newtonian, approximately |
| | | 600 mOsm |
| 4 | Methyl cellulose (0.5% w/v) | Polyethylene glycol 6000 |
| | in PBS, dynamic viscosity | (100 mM) in PBS, dynamic |
| | 14 mPa s, shear-thinning, | viscosity 32 mPa s, almost |
| | 285 mOsm | Newtonian, >1400 mOsm |

For all realisations listed in the table, well-defined separation boundaries form at the boundaries between the sample fluid and sheath fluid at total flow rates within a range of 3 nl/s to 400 nl/s, regardless of the flow rates used. This can clearly be seen by means of bright field microscopy, as shown in FIGS. 2, 3 and 5, and is even significantly more pronounced if phase contrast microscopy is used.

In the invention, the flow rates of the sample fluid 10 and sheath fluids 11 were adjusted independently of each other using pumps. This can also be achieved by means of pressure, electroosmosis, capillary forces and hydrostatics. Using the virtual channel resizing according to the invention, the width of the flow of sample fluid 10 can be adjusted, thus directly impacting its parabolic flow profile, as previously discussed.

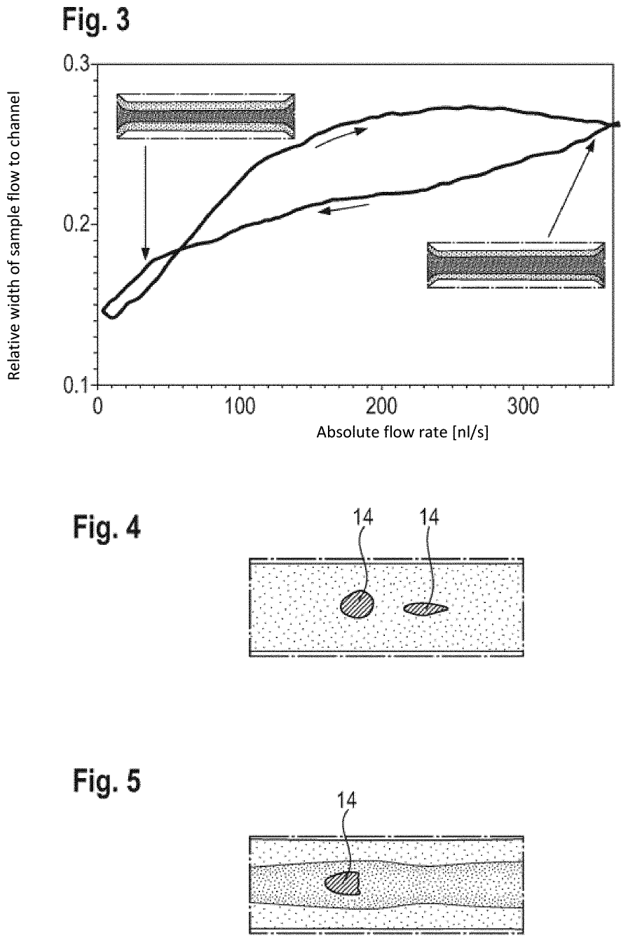

There are two possible ways of achieving this. Firstly, the ratio of the flow rates of the sheath fluids 11 and sample fluid 10 can be adjusted, as in the conventional RT-DC experiment according to WO 2015/024690 A1, in order to adjust the channelling of the sample fluid 10. It is important, however, that the flow profile in the microfluidic channel 12 is also dependent on the absolute flow rate. A corresponding effect is shown in FIG. 3, for example.

Secondly, the width of the flow of the sample fluid 10 can also be adjusted in virtual channel resizing by altering the composition of sample fluid and sheath fluid and the absolute channel width. The compositions influence the width of the flow across the dynamic viscosities. This behaviour is described in Li et al., equation [20].

FIG. 3 shows the behaviour of the relative width of the flow of sample fluid 10 in relation to the total width of the channel 12, respectively perpendicular to the direction of flow in the microfluidic channel 12 depending on the total or absolute flow rate. It is noticeable here that the behaviour as the flow rate increases (arrow pointing upwards to the right) is fundamentally different from the behaviour as the flow rate falls (arrow pointing downwards to the left). In particular, the ratio of the relative width of the flow of sample fluid 10 to the width of the channel 12 primarily plateaus as the flow rate rises above a certain flow rate (approximately 200 nl/s in the present case), whereas there is no such plateau as the flow rate falls. The experiment shown in FIG. 3 was performed with composition 1 (see table) in a microfluidic channel with a cross-section of 40 μm×40 μm. The relative width is of course dependent on the edge length of the channel cross-section, as verified or predicted by Li et al., J. Fluids Eng. 2011, 111202.

It can also be seen in FIG. 3 that it is possible using virtual channel resizing to cover a further range of virtual channel widths, wherein only the pumps specifically have to be controlled. It is neither necessary to change the solutions, nor do the samples have to be changed.

In the present example, the ratio of the flow of sample fluid 10 to the flow of sheath fluid 11 is 2 to 1 for all absolute flow rates shown in FIG. 3. The relative width of the flow of sample fluid 10 can vary between 14% (at 4 nl/s) and 27% (at 250 nl/s and on increase of the pump power). The two insets in FIG. 3 illustrate how this change can be mapped visually and show phase contrast images of the respective channels 12. The sample fluid 10 consists of 0.5 percent by weight methyl cellulose in PBS, the sheath fluid of 100 mM PEG8000 in PBS.

It is therefore possible to adjust the hydrodynamic conditions to the cells to be examined, as the channel diameter can respectively be adapted as desired to the diameter of the cells. To this extent, it is possible to use a microfluidic channel 12 which is significantly larger than the cells to be examined or the bodies to be examined and then to make the channel smaller virtually so that the channel matches the cells or bodies to be examined.

A hysteresis of the relative width of the flow of sample fluid 10 relative to the width of the microfluidic channel 12 depending on the control of the pumps can further be seen in FIG. 3. Over a large interval of absolute flow rates, the relative width of the flow of sample fluid 10 is greater when the pump power is increased than when it is reduced.

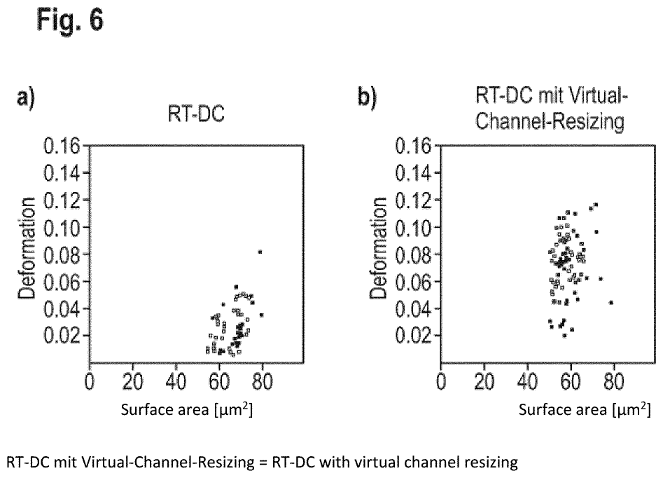

To illustrate the use of virtual channel resizing, the experiment on leucocytes in a whole blood measurement is shown in FIGS. 4 to 6 below. Once taken, the blood is stored in a citrate solution and diluted for the RT-DC assay in a methyl cellulose PBS solution at a ratio of 1 to 20 for the flow of sample fluid 10. FIG. 4 shows one granulocyte and one erythrocyte (on the left and right respectively) within a 40 μm×40 μm channel, wherein 0.6 percent by weight methyl cellulose is used in PBS, in other words wherein both the sheath fluid and sample fluid consist of the same solution. FIG. 6(a) shows a scatter plot of the leucocytes in the same test set-up. It can be seen from this scatter plot that the granulocytes only deform slightly.

FIGS. 5 and 6(b) show a similar experiment, with the material of the sheath fluid PEG 8000 (100 mmol) being in PBS. As can clearly be seen from FIG. 5, a leukocyte is clearly deformed along the direction of flow. It can be seen from the scatter plot in FIG. 6(b) that the leukocytes have a significantly increased deformation.

Consequently, it is clear that the virtually narrowed flow of sample fluid 10 leads to a significantly more marked deformation, in other words a shift in the representation shown in FIG. 6 upwards along the Y axis, in other words the cell population is divided into sub-populations. It is also apparent from the drawings in FIGS. 4 and 5 how pronounced the differences in form are.

It is clear from the examples that cells can be deformed by means of virtual channel resizing, irrespective of the respective size of the channel 12. A relevant flow behaviour is also shown in FIG. 4 of Li et al., wherein “Case II” in this figure corresponds in many aspects to the presently used system.

The present invention allows the width of the flow of sample fluid 10 to only be adjusted through the choice of materials of both the sheath fluid and sample fluid, the choice of volumetric flow rate of the two fluids (e.g. using corresponding syringe pumps), and through the cross-section of the channel 12. This is described in the equations [20] and [21] in Li et al., whereby equation [21] describes the shear tensions. The deformations then depend solely on a virtual channel width and the viscosity, in other words the width of the flow of sample fluid 10. Use of the invention is therefore not confined to microfluidic chips, and it can also be produced in other geometries. It is thus possible, using the respective sheath fluids and pump settings, to achieve the same effects in glass cuvettes or tubes which may have a larger diameter by up to a few millimetres but can also be filled with sample fluid and sheath fluid(s).

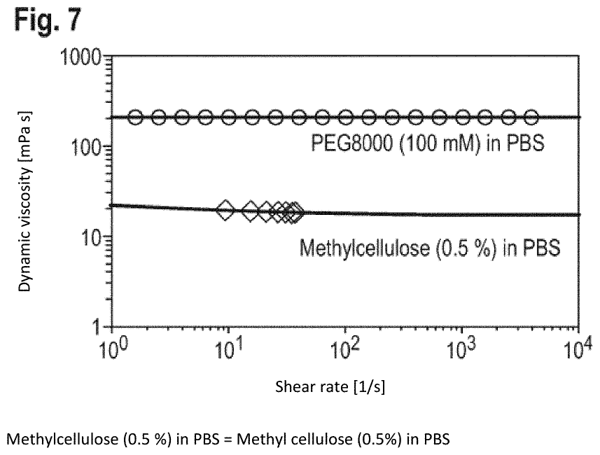

FIG. 7 further shows the rheological properties of the liquids used for the sheath fluid and the sample fluid. As can be seen from the figure, methyl cellulose in PBS manifests a slight shear-thinning behaviour, while PEG in PBS presents a primarily Newtonian behaviour. In particular, it is relevant that these two liquids manifest this behaviour across the relevant shear rate range (1 1/s-10,000 1/s).

As part of the present invention, the respective dynamic viscosities in the sheath fluid are equal to or greater than the dynamic viscosity of the sample fluid. However, it would, in principle, also be conceivable for the viscosity ratio to be chosen the other way around and for cells or bodies to still be deformed, even if such a design is currently regarded as being less advantageous.

A further aspect of the invention relates to the use of the method according to the invention for feeding substances into deformable bodies, in particular cells, wherein a substance to be fed in is contained in the sheath fluid and wherein parts of the deformable body are in contact with the sheath fluid, whereby this substance merges into the body owing to this contact. It is preferred here that the surface via which the deformable body is in contact with the sheath fluid is controlled and that the period of time during which the deformable body is in contact with the sheath fluid is controlled in order to control the amount of substance fed. Such a method allows the controlled feeding of substances, in particular medications, into deformable bodies, in particular cells.

1. A method for deforming deformable bodies, preferably droplets or cells, comprising the following steps:
feeding a sample fluid into a channel to create a laminar flow of the sample fluid, wherein the sample fluid transports deformable bodies,
feeding a sheath fluid into the channel to create a laminar flow of the sheath fluid such that the sheath fluid directly borders the sample fluid in a border region of the channel and flows in the same direction as the sample fluid at least in the border region,
wherein at the shear rates of the sheath fluid and sample fluid occurring in the channel the viscosity of the sheath fluid is greater than the viscosity of the sample fluid,
wherein the deformable bodies are deformed by the forces arising in the sample fluid in the channel due to the border region.

2. The method according to claim 1, wherein the dynamic viscosity of the sheath fluid in the border region is within a range of 1 mPa s to 1 Pa s, preferably of 50 mPa s to 250 mPa s and/or wherein the dynamic viscosity of the sample fluid in the border region is within a range of 1 mPa s to 100 mPa s, preferably of 5 mPa s to 50 mPa s.

3. The method according to claim 1, wherein the average flow rate of the sample fluid is within a range of 0.1 cm/s to 10 m/s and/or wherein the average flow rate of the sheath fluid is within a range of 0.1 cm/s to 10 m/s.

4. The method according to claim 1, wherein the sample fluid is a shear-thinning liquid containing the deformable bodies.

5. The method according to claim 1, wherein the sample fluid and the sheath fluid consist of liquids which do not mix, or only do so to a minor extent, at least within the time scale during which the deformable bodies traverse the border region.

6. The method according to claim 1, wherein the sheath fluid is a Newtonian or shear-thickening liquid.

7. A method for determining the mechanical properties of deformable bodies, preferably cells, wherein deformable bodies are deformed using the method according to claim 1 and wherein the deformation of the deformable bodies is preferably measured using an optical method.

8. A method for examining the cell biological properties of cells, comprising the deformation of cells with the method according to claim 1 and the examination of the biochemical properties of cell components, in particular the cytoskeleton or organelles, based on changes in these cell components owing to deformation, wherein the changes are preferably measured using a fluorescence-based method, in particular flow cytometry.

9. A method for sorting deformable bodies, in particular cells, comprising:
performing the method according to claim 7 and
sorting cells based on the particular mechanical and/or cell biological properties, preferably with a flow cytometer with a sorting function.

10. The method according to claim 1, wherein prior to the start of measuring the deformation of the deformable bodies the total flow rate of the sample fluid and sheath fluid is increased from an initial value to a higher target value.

11. A device which is designed to perform the method according to claim 1.
